(19) United States
(12) Patent Application Publication
(10) Pub. No.: US 2008/0311059 A1
(43) Pub. Date: Dec. 18, 2008

(54) COMPOSITION FOR SKIN CARE AND METHOD FOR THE SAME

(75) Inventors: Yung-Sheng LIN, Changhua County (TW); Ming-Yu LIN, Hsinchu City (TW); Tsung-Tao Huang, Banciao City (TW); Ting-Kai Leung, Taipei City (TW); Chwen-Ming SHIH, Sihih City (TW)

Correspondence Address: VOLPE AND KOENIG, P.C., UNITED PLAZA, SUITE 1600, 30 SOUTH 17TH STREET, PHILADELPHIA, PA, 19103, US

(73) Assignees: NATIONAL APPLIED RESEARCH LABORATORIES, Taipei (TW); TAIPEI MEDICAL UNIVERSITY, Taipei City (TW)

(21) Appl. No.: 11/857,158

(22) Filed: Sep. 18, 2007

(30) Foreign Application Priority Data
Jun. 15, 2007 (TW) 096121948
Jun. 15, 2007 (TW) 096121951

(51) Int. Cl. A61K 8/26; A61K 8/22; A61Q 17/04; A61Q 19/00

(52) U.S. Cl. 424/59; 424/62

(57) ABSTRACT

A skin whitening composition and a method for the same are provided. The skin whitening composition comprises a far-infrared ray releasing substance having a primary component of an oxide mineral, wherein the skin whitening composition achieves a whitening effect via an irradiation of the far-infrared ray releasing substance. In another aspect, the method comprises the steps of administering a far-infrared ray releasing substance to a subject and reducing melanogenesis of the subject by inhibiting growth of a pigment cell, wherein the far-infrared releasing substance has an oxide mineral.

### FIELD OF THE INVENTION

The present invention relates to a composition and a method for skin care through a far-infrared ray releasing substance.

### BACKGROUND OF THE INVENTION

Melanogenesis is a common physical phenomenon when skin is exposed under UV or other light sources. When the skin is stimulated, the melanin will be produced by melanocytes and sent to epithelial cells to darken the skin.

Whitening is an important appreciation of the beauty, and there are plenty of whitening products in the market. Among these whitening products, some have efficiency by far-infrared ray irradiation. However, most whitening products supplemented with the far-infrared ray releasing substance emphasize on the effect of promoting whitening ingredients to be absorbed by the skin rather than the whitening effect of the far-infrared ray releasing substance itself.

According to International Commission on Illumination (CIE1987), the far-infrared ray (FIR) is an electromagnetic wave with the wavelength of 3-1000 μm. Among them, the far-infrared ray having the wavelength of 3-14 μm is called the light of life, because of its advantages in the growth of animals and plants. Currently, it has been proved that FIR has therapeutic effect on many human diseases, and thus is often applied on many physiological purposes, for example, blood circulation acceleration, metabolism activation, tissue regeneration and immune system activation, etc. However, the effect on melanogenesis of the far-infrared ray has not been investigated.

Currently, most of the whitening products promote the skin whitening chemically by using various active ingredients; nevertheless, they may lead to swelling or decortication resulting from allergy or other side effects. However, the present invention provides a composition and a method for skin care that reduce melanogenesis and promote skin whitening by the physical character of the far-infrared ray. Therefore, the physical method for skin care can exhibit a whitening effect and reduce the side effects by the far-infrared ray releasing substance alone, or improve the effect of other whitening ingredients while accompanied with the other whitening ingredients. Hence, it is guaranteed that the users will be safe and healthy.

In view of the drawbacks of current techniques, the inventors develop a composition and a method for skin care that reduce melanogenesis by a far-infrared ray releasing substance. The summary of the present invention is described below.

### SUMMARY OF THE INVENTION

It is an aspect of the present invention to provide a far-infrared cream for skin care comprising a 1-20% far-infrared ray (FIR) releasing substance including an oxide mineral 80%-99.9% in weight, wherein the oxide mineral comprises an aluminum oxide 60-95% in weight.

Preferably, the far-infrared cream further comprises at least one selected from a group consisting of an active ingredient, an adjuvant and an additive 80-99% in weight.

In one preferred embodiment, the far-infrared cream is a cosmetic.

Preferably, the cosmetic further comprises at least one cosmetic material selected from a group consisting of an oil, a fat, a wax, a hydrocarbon, a fatty acid, a sterol, a hormone, a vitamin, a fatty acid ester, a humectant, surfactant, a color, a viscosity improver, a film former, an UV absorber, an antifungal, an anti-inflammatory agent, an antiperspirant, an antiseptic and an aromatic.

In another preferred embodiment, the far-infrared cream is a pharmaceutical composition.

Preferably, the pharmaceutical composition further comprises a pharmaceutically acceptable carrier.

In still another preferred embodiment, the far-infrared cream is an industrial product.

It is another aspect of the present invention to provide a skin whitening composition comprising a far-infrared ray releasing substance having a primary component of an oxide mineral, wherein the skin whitening composition achieves a whitening effect via an irradiation of the far-infrared ray releasing substance.

Preferably, the skin whitening composition further comprises at least one selected from a group consisting of an active ingredient, an adjuvant and an additive.

In one preferred embodiment, the skin whitening composition is a cosmetic.

In another preferred embodiment, the skin whitening composition is a pharmaceutical composition.

Preferably, the oxide mineral comprises an aluminum oxide, for example, 60-95% aluminum oxide.

Preferably, the mineral oxide comprises a 1-20% ferric oxide, a 1-10% magnesium oxide and a 1-30% calcium carbonate.

In still another preferred embodiment, the skin whitening composition further comprises a base material selected from a group consisting of a metal, a glass, a ceramic and a polymer, wherein the base material is mixed with the far-infrared releasing substance.

It is a further aspect of the present invention to provide a method for whitening a skin of a subject. In an exemplary embodiment, the method comprises the steps of administering a far-infrared ray releasing substance to the subject and reducing melanogenesis of the subject by inhibiting growth of a pigment cell, wherein the far-infrared ray releasing substance has an oxide mineral.

Preferably, the oxide mineral is an aluminum oxide.

Preferably, the aluminum oxide is 60-95% in weight.

According to one preferred embodiment, the method further comprises steps of providing the far-infrared ray releasing substance as an ingredient of a cosmetic and administering the far-infrared ray releasing substance to the subject by one of a smearing and topical applications.

According to another preferred embodiment, the method further comprises a step of mixing the far-infrared ray releasing substance with a base material being selected from a group consisting of a metal, a glass, a ceramic and a polymer for the administration.

Other objects, advantages and efficacies of the present invention will be described in detail below taken from the preferred embodiments with reference to the accompanying drawings, in which:

### BRIEF DESCRIPTION OF THE DRAWINGS

FIG. 1 is a diagram showing a preferred embodiment of the far-infrared base material of the present invention;

FIGS. 2(a)-2(b) are bar graphs showing the effect of the FIR releasing substance of the present invention on the growth of pigment cells (B16F10), wherein the FIR represents the group of the FIR releasing substance whereas the milk powder represents the control group, and FIG. 2(a) and FIG. 2(b) represent the results of 24 hrs and 48 hrs, respectively;

FIGS. 3(a)-3(b) are bar graphs showing the effect of the FIR releasing substance of the present invention on the melanogenesis, wherein FIR represents the group of the FIR releasing substance whereas the milk powder represents the control group, and FIG. 3(a) and FIG. 3(b) represent the results of 24 hrs and 48 hrs, respectively; and

FIGS. 4(a)-4(b) are bar graphs showing the effect of the FIR releasing substance of the present invention on the activity of tyrosinase in pigment cells, wherein FIR represents the group of the FIR releasing substance whereas the milk powder represents the control group, and FIG. 4(a) and FIG. 4(b) represent the absorption at 370 nm and 560 nm, respectively.

### DETAILED DESCRIPTION OF THE PREFERRED EMBODIMENT

### Example I

### The Far-Infrared Ray (FIR) Releasing Cream

The FIR releasing substance of the present invention is a high-efficiency far-infrared ray ceramic powder developed by the Instrument Technology Research Center of the National Applied Research Laboratories and the Taipei Medical University. The biological effect of the FIR releasing substance is proved by many experiments, and the composition thereof is composed of various natural minerals. These natural minerals in the FIR releasing substance are about 80-99.9% in weight, and comprise 60-95% aluminum oxide, 1-20% ferric oxide, 1-10% magnesium oxide and 1-30% calcium carbonate. Besides, the FIR releasing substance further comprises other ingredients including titanium dioxide, titanium boride and more natural minerals such as silicon oxide, zinc hydroxide, zinc oxide and carbides, etc. The average emissivity of the FIR releasing substance is over 0.98 at a wavelength between 6-14 μm, which is measured by an FIR spectrometer using a black body as a standard. Furthermore, the far-infrared ray released by the FIR releasing substance has an anti-bacterial rate of over 99.9% against Staphylococcus and Escherichia coli according to the AATCC 100 standard.

The present invention relates to an FIR cream, which is composed by mixing an FIR releasing substance of different percentages with a cosmetic cream. Preferably, the FIR cream comprises 1-20% FIR releasing substance in weight. The cosmetic cream comprises at least one cosmetic material selected from a group consisting of oil, fat, wax, hydrocarbon, fatty acid, sterol, hormone, vitamin, fatty acid ester, humectant, surfactant, color, viscosity improver, film former, UV absorber, antifungal, anti-inflammatory agent, antiperspirant, antiseptic and aromatic. In order to increase the efficacy, the FIR cream also comprises other ingredients, adjuvants and additives. It is proved by a standard experiment that the FIR cream of the present invention would not cause the skin allergies, and hence, the FIR cream shall have a broad market in skin care and whitening.

The FIR cream in this embodiment is a cosmetic of topical application for whitening the skin of a subject, improving blood circulation, promoting metabolism, improving the constitution of a subject and improving the health of a subject. However, the FIR cream is not limited to a cosmetic; it can be a pharmaceutical composition or an industrial product. Alternately, the FIR cream of the present invention is an industrial product such as the paint, coating, lubricant, film, gel, latex, building material, ink, dye, wax and polish. As long as a user is located in an environment equipped with the mentioned industrial products, the industrial products can bring their whitening effect and provide an easier method for skin care.

### Example II

### The Far-Infrared Ray (FIR) Base Material

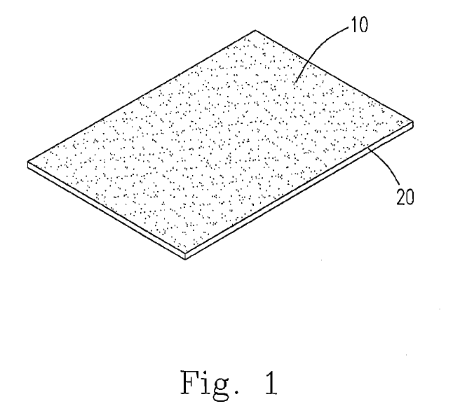

Please refer to FIG. 1, which is a diagram showing a preferred embodiment of the far-infrared base material of the present invention. As FIG. 1 shows, the far-infrared ray base material comprises a base material 20 and an FIR releasing substance 10 mixed with the base material 20. The base material 20 is selected from a group consisting of a metal, a glass, a ceramic and a polymer. The far infrared ray releasing substance 10 is in a form selected from a group consisting of a bulk solid, a grain, a powder and a membrane. The other characters of the FIR releasing substance 10 such as the ingredient and the efficacy are identical to the first embodiment.

The application of the FIR base material 20 is various, especially the use of skin whitening. The FIR base material 20 can be made as many common devices, medical devices, building materials, transportations and fabrics for skin whitening at liberty.

### Example III

### The Composition for Skin Care

The third embodiment of the present invention is a composition for skin care, which comprises an FIR releasing substance, wherein the other characters of the FIR releasing substance such as the ingredient and the efficacy are identical to the first embodiment. The composition achieves its whitening effect through the far-infrared ray released by the FIR releasing substance. For being more effective, the composition herein further comprises other active ingredients, adjuvant and additives. As to the form of the composition, it can appear as a lotion, a cream, a gel, a solution, a gel dispersed solution, a suspension, an ointment, a cleaning agent, a foam or a Latex foam. If the composition is a cosmetic, it could comprise some cosmetic materials including oil, fat, wax, hydrocarbon, fatty acid, sterol, hormone, vitamin, fatty acid ester, humectant, surfactant, color, viscosity improver, film former, UV absorber, antifungal, anti-inflammatory agent, antiperspirant, antiseptic and aromatic. Alternately, if the composition is a pharmaceutical composition, it further comprises a pharmaceutically acceptable carrier, an excipient and a diluent for skin whitening by different routes.

### Example IV

### The Effect of the Fir Releasing Substance on Inhibiting Growth of B16-F10 Cells

Equal amounts of 450 cm³ FIR powder (FIR groups) and nonfunctional milk powder (control groups) were enclosed by different bags, which are made of synthetic or natural high polymer, metal, glass or ceramics, etc.

1×10⁴ B16-F10, a melanocyte, are grown in the DMEM medium with 10% fetal calf serum and incubated in a 5% CO₂ incubator at 37° C. The bags filled with FIR powder (as the FIR groups) and the bags filled with non-functional powder (as the control group) were inserted beneath the dishes of B16-F10 cells, which are cultured with the cells without direct contact. After 24 hrs and 48 hrs, the MTT solution (3-(4,5-dimethylthiazol-2-yl)-2,5-diphenyltetrazolium bromide) is added into the cultured cells for a 4-hr incubation. Subsequently, the suspension of the culturing is removed and the DMSO (dimethyl sulfoxide) is added thereinto. After a 10-minute incubation, the absorbance of the cells is measured at 540 nm.

Result

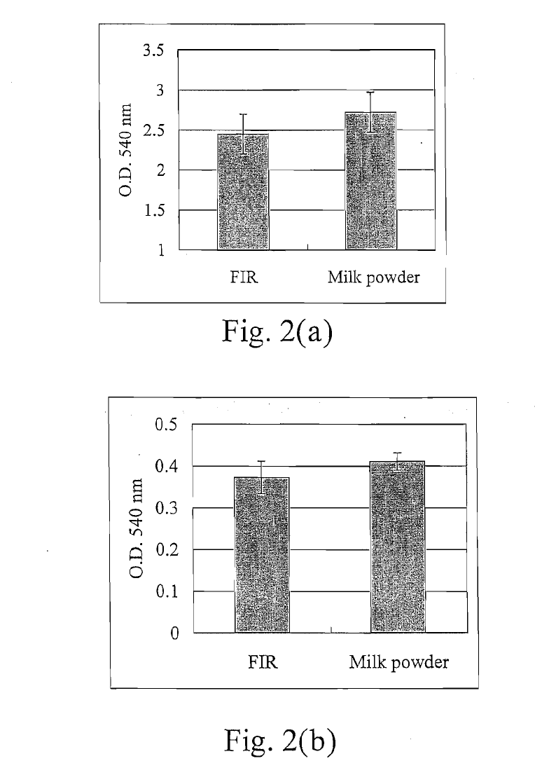

Please refer to FIG. 2(a) and FIG. 2(b), which show the effect of the FIR releasing substance of the present invention on the growth of pigment cells (B16F10). FIG. 2(a) and FIG. 2(b) represent the results of cell growth co-cultured with the FIR releasing substance for 24 hrs and 48 hrs, respectively. The milk powder serves as a control group for comparing the difference between the control group and the FIR group. As FIG. 2(a) illustrates, the FIR releasing substance of the present invention has an inhibition to the growth of the melanoma. The FIR group apparently decreases the growth of the melanoma to 10% and 9.7% than the control groups in 24 hrs and 48 hrs, respectively. According to the above results, the FIR releasing substance of the present invention inhibits the growth of the melanoma efficiently, and thus decreases the source of the melanin. Therefore, it is proved that the FIR releasing substance has an effect for skin whitening through affecting the growth of the melanocytes.

### Example V

### The Effect of the Fir Releasing Substance on Decreasing Melanin in B16-F10 Cells

The B16-F10 cells are cultured as the above condition and incubated in the incubator for being used for experiments, and the cells are cultured with the FIR releasing substance and the milk powder as the above procedure. After 24 hrs and 48 hrs, the cells are tripsinized from the culturing dish and washed with phosphate buffered saline (PBS) once. The detached cells are transferred to a 1.5 ml microtube and centrifuged at 5000 rpm for 5 minutes. After the suspension is removed, 1 ml of the solution containing 90% 1M sodium hydroxide and 10% DMSO is added into the microtube, and the microtube is heated at 80° C. for 1 hr. Finally, the absorbance of the cells is measured at 475 nm.

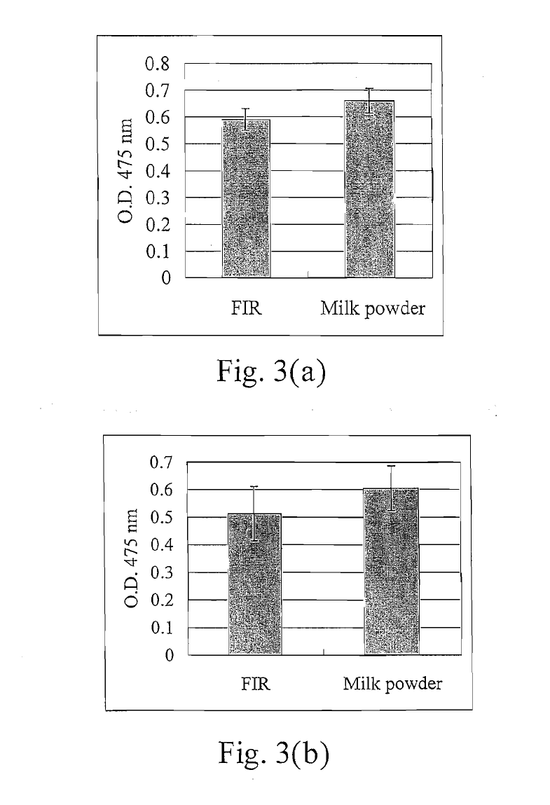

Please refer to FIG. 3(a) and FIG. 3(b), which show the amount of melanin in B16-F10 cells cultured for 24 hrs and 48 hrs, respectively. After being co-cultured with the B16-F10 cells for 24 hrs and 48 hrs, the FIR releasing substance decreases melanin produced in the B16-F10 cells to 11% and 16% in comparison with the control group, respectively. This result matches that of the cell growth, and further proves the inhibition to melanin production of the FIR releasing substance. Therefore, it is known that the FIR releasing substance can be applied to the skin of a subject for reducing the melanin in the skin.

### Example VI

### The Effect of the Fir Releasing Substance on Decreasing Tyrosinase Activity in B16-F10 Cells

Two 6-well culturing plates containing 1×10⁴ B16-F10 cells in each well are cultured according to the above condition. Equal amounts of 450 cm³ FIR powder and nonfunctional milk powder are placed beneath the two culturing dishes respectively. After 24 hrs, the cells are tripsinized and centrifuged at 6000 rpm for 5 minutes for removing the suspension. A 150 μl solution containing 0.1 M PBS (pH value 6.8) and 1% Triton X-100 is added into the cell pallet and the mixture is then incubated at room temperature. 30 minutes later, the mixture is centrifuged at 13000 rpm for 30 minutes to obtain a suspension. 50 μl of suspension is taken to be filled into a predetermined well of a 96-well plate for an 1-hr incubation at room temperature. Then, the suspension of the 96-well plate is removed and the plate is washed three times with PBS. A 200 μl PBS containing 5% skim milk powder is added into the plate for blocking at room temperature. After 1 hr, the 96-well plate is washed three times with a PBST solution containing 0.1% surfactant, Tween-20, in the PBS. A 100 μl of mouse anti-human tyrosinase antibody with 50 times dilution is used as the first antibody and added into each well of the plate at room temperature. After an 1-hr reaction, the plate is washed with PBST solution for three times, and a 100 μl of goat anti-mouse antibody with 5000 times dilution serving as the second antibody is added into the plate at room temperature. After an 1-hr reaction, the plate is washed with PBST solution for three times. Finally, a 200 μl of tetramethylbenzidine (TMB) solution is added into the plate for reaction in the dark. 30 minutes later, the absorbance of each well of the plate is measured at 370 nm and 650 nm respectively.

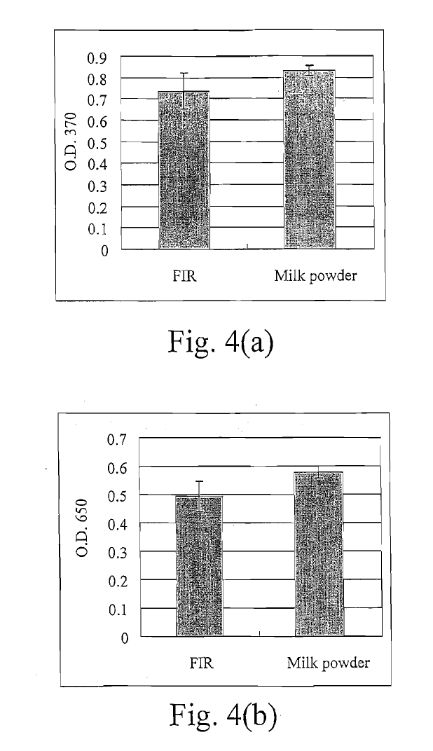

Please refer to Table 1, Table 2, FIG. 4(a) and FIG. 4(b). Table 1 and Table 2 illustrate the results of measurement at 370 nm and 650 nm respectively. FIG. 4 (a) and FIG. 4(b) show the bar graphs of Table 1 and Table 2, respectively. FIR represents the group of the FIR releasing substance whereas the milk powder represents the control group, and the absorbance of the FIR group is less than the control group whether at 370 nm or 650 nm. It is known that the FIR releasing substance inhibits the activity of the tyrosinase in melanocytes, and thus the tyrosinase can not catalyze the tyrosin as melanin. According to the above result, it is resonable that the FIR releasing substance existing in a cosmetic suppresses the melanin produced by tyrosinase in the skin and prevents darkening of the skin. Therefore, the FIR releasing substance certainly can be an ingredient of a cosmetic and be mixed with other materials for providing the whitening effect.

TABLE 1

| 370 nm | FIR | Milk powder |
|---|---|---|
| 1 | 0.63 | 0.853 |
| 2 | 0.742 | 0.847 |
| 3 | 0.836 | 0.801 |
| Average | 0.736 | 0.834 |

TABLE 2

| 650 nm | FIR | Milk powder |
|---|---|---|
| 1 | 0.432 | 0.598 |
| 2 | 0.485 | 0.589 |
| 3 | 0.564 | 0.549 |
| Average | 0.494 | 0.579 |

To summarize, the present invention proposes a composition and a method for skin care, which achieves the whitening effect directly through a far-infrared ray releasing substance, where the far-infrared ray releasing substance is used to promote the effect of other ingredients of the cosmetic in the prior art. Further, the present invention provides a method for skin care, which administers a far-infeared ray releasing substance to a subject, and reducing melanogenesis of the subject by inhibiting growth of a pigment cell. Thus, the present invention not only bears novelty and obviously progressive nature, but also bears the utility for the industry.

While the invention has been described in terms of what is presently considered to be the most practical and preferred embodiments, it is to be understood that the invention needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. A far-infrared cream for skin care, comprising:
a 1-20% far-infrared ray releasing substance including an oxide mineral 80%-99.9% in weight, wherein the oxide mineral comprises an aluminum oxide 60-95% in weight.

2. The far-infrared cream as claimed in claim 1, further comprising:
at least one selected from a group consisting of an active ingredient, an adjuvant and an additive 80-99% in weight.

3. The far-infrared cream as claimed in claim 1, wherein the far-infrared cream is a cosmetic.

4. The far-infrared cream as claimed in claim 3, wherein the cosmetic further comprises at least one cosmetic material selected from a group consisting of an oil, a fat, a wax, a hydrocarbon, a fatty acid, a sterol, a hormone, a vitamin, a fatty acid ester, a humectant, a surfactant, a color, a viscosity improver, a film former, an UV absorber, an antifungal, an anti-inflammatory agent, an antiperspirant, an antiseptic and an aromatic.

5. The far-infrared cream as claimed in claim 1, wherein the far-infrared cream is a pharmaceutical composition.

6. The far-infrared cream as claimed in claim 5, wherein the pharmaceutical composition further comprises a pharmaceutically acceptable carrier.

7. The far-infrared cream as claimed in claim 1, wherein the far-infrared cream is an industrial product.

8. A skin whitening composition, comprising:
a far-infrared ray releasing substance having a primary component of an oxide mineral,
wherein the skin whitening composition achieves a whitening effect via an irradiation of the far-infrared ray releasing substance.

9. The skin whitening composition as claimed in claim 8, further comprising:
at least one selected from a group consisting of an active ingredient, an adjuvant and an additive.

10. The skin whitening composition as claimed in claim 8, wherein the skin whitening composition is a cosmetic.

11. The skin whitening composition as claimed in claim 8, wherein the skin whitening composition is a pharmaceutical composition.

12. The skin whitening composition as claimed in claim 8, wherein the oxide mineral comprises an aluminum oxide.

13. The skin whitening composition as claimed in claim 12, wherein the aluminum oxide is 60-95% in weight.

14. The skin whitening composition as claimed in claim 8, wherein the mineral oxide comprises a 1-20% ferric oxide, a 1-10% magnesium oxide and a 1-30% calcium carbonate.

15. The skin whitening composition as claimed in claim 8, further comprising:
a base material selected from a group consisting of a metal, a glass, a ceramic and a polymer,
wherein the base material is mixed with the far-infrared releasing substance.

16. A method for whitening a skin of a subject, comprising the steps of:
administering a far-infrared ray releasing substance to the subject, wherein the far-infrared ray releasing substance has an oxide mineral; and
reducing melanogenesis of the subject by inhibiting growth of a pigment cell.

17. The method as claimed in claim 16, wherein the oxide mineral is an aluminum oxide.

18. The method as claimed in claim 17, wherein the aluminum oxide is 60-95% in weight.

19. The method as claimed in claim 16, further comprising steps of:
providing the far-infrared ray releasing substance as an ingredient of a cosmetic; and
administering the far-infrared ray releasing substance to the subject by one of a smearing and topical applications.

20. The method as claimed in claim 16, further comprising a step of:
mixing the far-infrared ray releasing substance with a base material being selected from a group consisting of a metal, a glass, a ceramic and a polymer for the administration.
